(19) United States
(12) Patent Application Publication
(10) Pub. No.: US 2006/0234264 A1
(43) Pub. Date: Oct. 19, 2006

(54) Multiplex polynucleotide synthesis

(75) Inventor: Paul Hardenbol, San Francisco, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/375,818

(22) Filed: Mar. 14, 2006

Related U.S. Application Data
(60) Provisional application No. 60/662,032, filed on Mar. 14, 2005.

(51) Int. Cl. C12Q 1/68; C12P 19/34

(52) U.S. Cl. 435/6; 435/91.2

(57) ABSTRACT

The invention provides a method of synthesizing complex mixtures of long polynucleotides by separately synthesizing and assembling shorter component oligonucleotides. In one aspect, pairs of oligonucleotides that form components of such polynucleotides are synthesized on one or more microarrays, or other large-scale parallel solid phase synthesis platforms, after which they are released. Members of each pair contain unique complementary barcode sequences that are used match-up pairs in a hybridization reaction to form duplexes. Such duplexes are then extended with a DNA polymerase and the resulting extension product is amplified to form an amplicon. The amplicon may be either used directly as the desired polynucleotide, or it may undergo further processing, such as capture on solid phase supports and/or additional enzymatic or chemical processing, to produce a desired polynucleotide product, such as a circularizing probe for multiplex analysis of genomic DNA, or the like.

### RELATED APPLICATIONS

This application claims priority to provisional application No. 60/662,032 filed Mar. 14, 2005, the entire disclosure of which is incorporated herein by reference.

### FIELD OF THE INVENTION

The present invention relates to methods for synthesizing mixtures of nucleic acids, and more particularly, for synthesizing multiplexed nucleic acid probes.

### BACKGROUND

The use of complex mixtures of nucleic acid probes has increased as more and more large-scale genetic studies have taken place, which are designed to interrogate many thousands of genetic loci at the same time, Hardenbol et al, Nature Biotechnology, 21: 673-678 (2003; Fan et al, Genome Research, 10: 853-860 (2000); Chen et al, Genome Research, 10: 549-557 (2000); Hirschhorn et al, Proc. Natl. Acad. Sci., 97: 12164-12169 (2000); Lashkari et al, Proc. Natl. Acad. Sci., 94: 8945-8947 (1997). The production of complex mixtures of such probes can be expensive and labor-intensive if each probe is synthesized separately and then combined in the proper amounts for use. There have been attempts to address this problem by making use of oligonucleotides that are synthesized in parallel on microarrays, or like supports, e.g. Weiler et al, Anal. Biochem., 243: 218-227 (1996); Frank et al, Nucleic Acids Research, 11: 4365-4377 (1983); Lipschutz et al, U.S. Pat. No. 6,440,677. However, such approaches have not been practical for a variety of reasons, including poor and/or variable yields of individual species, unbalanced representation of the various sequences in a mixture, and difficulties in making sufficient quantities of polynucleotides for performing hybridization reactions.

The availability of methods of synthesizing mixtures of polynucleotides that overcome the deficiencies of prior art would greatly improve research, medical, and industrial applications that require large-scale multiplex or parallel analysis with hybridizations probes.

### SUMMARY OF THE INVENTION

The invention provides a method of synthesizing complex mixtures of long polynucleotides by separately synthesizing and assembling shorter component oligonucleotides. In one aspect, pairs of oligonucleotides that form components of such polynucleotides are synthesized on one or more microarrays, or other large-scale parallel solid phase synthesis platforms, after which they are cleaved from the supports. Members of each pair contain unique complementary barcode sequences that are used match-up pairs in a hybridization reaction to form duplexes. Such duplexes are then extended with a DNA polymerase and the resulting extension product is amplified to form an amplicon. The amplicon may be either used directly as the desired polynucleotide, or it may undergo further processing, such as capture on solid phase supports and/or additional enzymatic or chemical processing, to produce a desired polynucleotide product, such as a circularizing probe for multiplex analysis of genomic DNA, or the like.

In one aspect, the method of the invention comprises the following steps: (a) synthesizing a plurality of first oligonucleotides on a first microarray, each first oligonucleotide having a predetermined sequence comprising in the 3′ to 5′ direction a first barcode sequence, a first variable region, and a first primer binding site; (b) synthesizing a plurality of second oligonucleotides on a second microarray, each second oligonucleotide having a predetermined sequence comprising in the 3′ to 5′ direction a second barcode sequence, a second variable region, and a second primer binding site, the second barcode sequences being selected so that for every first barcode sequence there is at least one second barcode sequence complementary thereto; (c) cleaving the first oligonucleotides and the second oligonucleotide from the first and second microarrays so that such cleaved first oligonucleotides and second oligonucleotides have extendable 3′ ends; (d) mixing the cleaved first oligonucleotides and second oligonucleotides under conditions that permit the formation of stable duplexes substantially only between first barcode sequences and complementary second barcode sequences; (e) extending 3′ ends of the stable duplexes with a DNA polymerase to form a mixture of polynucleotides, each polynucleotide of the mixture having first and second primer binding sites. The first microarray and second microarray may be the same or different solid phase supports. In one aspect of the invention, barcode sequences are members of a minimally cross-hybridizing set of oligonucleotides to enhance the specificity of hybridizations forming duplexes for extension. That is, barcode sequences are selected so that under stringent hybridization conditions, substantially only perfectly matched duplexes form between barcode sequences and their respective complements. In some aspects, one or the other of the first and second variable regions may be absent, so that the polynucleotides formed after the step of extending have only a single variable region.

In another aspect, the method of the invention further includes the following steps: amplifying the extended polynucleotides from the hybridization reaction to form an amplicon, removing the first and second primer binding sites from at least one strand of the amplicon, and isolating the polynucleotide of interest. In one embodiment, the step of amplifying may be carried out with a polymerase chain reaction (PCR) using at least one primer that is specific for either the first primer binding site or the second primer binding site and that has a capture moiety attached, such as biotin. In further embodiments of the invention, the step of isolating the polynucleotide may be accomplished by capturing its associated amplicon on a solid phase support by the capture moiety. After such capture, the first and second primer binding sites may be cleaved from a strand of the amplicon, e.g. using nicking enzymes, and the desired polynucleotides may be separated from such reaction mixture.

The invention provides advances over prior approaches by providing preparative-scale mixtures of polynucleotides assembled from component oligonucleotides efficiently synthesized on an analytical-scale on highly parallel synthesis platforms, such as microarrays. Such polynucleotide mixtures are highly useful in constructing hybridization probes for large-scale genetic measurements.

### BRIEF DESCRIPTION OF THE FIGURES

FIGS. 1A-1C illustrate one embodiment for synthesizing first and second oligonucleotide mixtures and their use to form a polynucleotide mixture of the invention.

FIGS. 2A-2B show an application of polynucleotide mixtures of the invention for making circularizable probes.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451;683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

### DEFINITIONS

“Addressable” in reference to tag complements means that the nucleotide sequence, or perhaps other physical or chemical characteristics, of an end-attached probe, such as a tag complement, can be determined from its address, i.e. a one-to-one correspondence between the sequence or other property of the end-attached probe and a spatial location on, or characteristic of, the solid phase support to which it is attached. Preferably, an address of a tag complement is a spatial location, e.g. the planar coordinates of a particular region containing copies of the end-attached probe. However, end-attached probes may be addressed in other ways too, e.g. by microparticle size, shape, color, frequency of micro-transponder, or the like, e.g. Chandler et al, PCT publication WO 97/14028.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a gene or portion of a gene in a genome, or it may refer to any contiguous portion of genomic sequence whether or not it is within, or associated with, a gene. Preferably, a genetic locus refers to any portion of genomic sequence from a few tens of nucleotides, e.g. 10-30, in length to a few hundred nucleotides, e.g. 100-300, in length.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discernable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified.

“Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

As used herein, the term “Tₘ” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

Multiplex Polynucleotide Synthesis

In one aspect, the invention provides an efficient and economical method for producing complex hybridization probes that may be employed in a variety of analytical techniques, such as those described below. An important feature of the invention is the use of large scale parallel synthesis technologies, particularly microarrays, to efficiently synthesize oligonucleotide components that are assembled into complex mixtures of polynucleotide probes. As explained more fully below, the invention is particularly useful for synthesizing probes that comprise oligonucleotide tags, or barcodes, that have a one-to-one correspondence with (and forms a linear molecule with) a probe sequence that specifically hybridizes to a complementary target sequence in a sample. Such probes include, but are not limited to, molecular inversion probes (MIPs), e.g. Willis et al, U.S. Pat. No. 6,858,412; padlock probes, e.g. Landegren et al, U.S. Pat. No. 5,871,921; probes for multiplex ligation-dependent probe amplification (MLPA), e.g. Schouten, U.S. Pat. No. 6,955,901; selector probes, e.g. Dahl et al, Nucleic Acids Research, 33: e71 (2005); and the like. In accordance with one aspect of the invention, pairs of single stranded oligonucleotide components are synthesized such that one member of each pair contains a barcode sequence and the other member of the pair contains the complement of such sequence. Mixtures of such oligonucleotide may then be converted into duplexes by selecting conditions under which the barcode sequences and their respective complements form stable hybrids. 3′ ends of such duplexes may then be extended and the resulting duplex amplified using conventional techniques, after which the desired polynucleotide probes may be extracted.

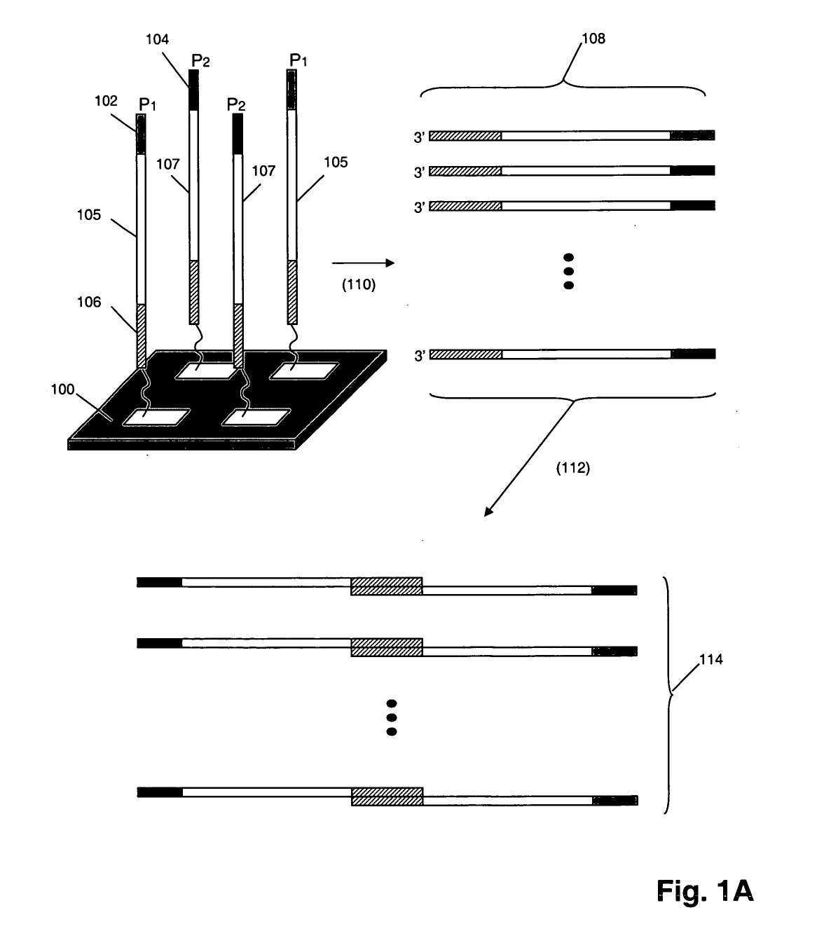

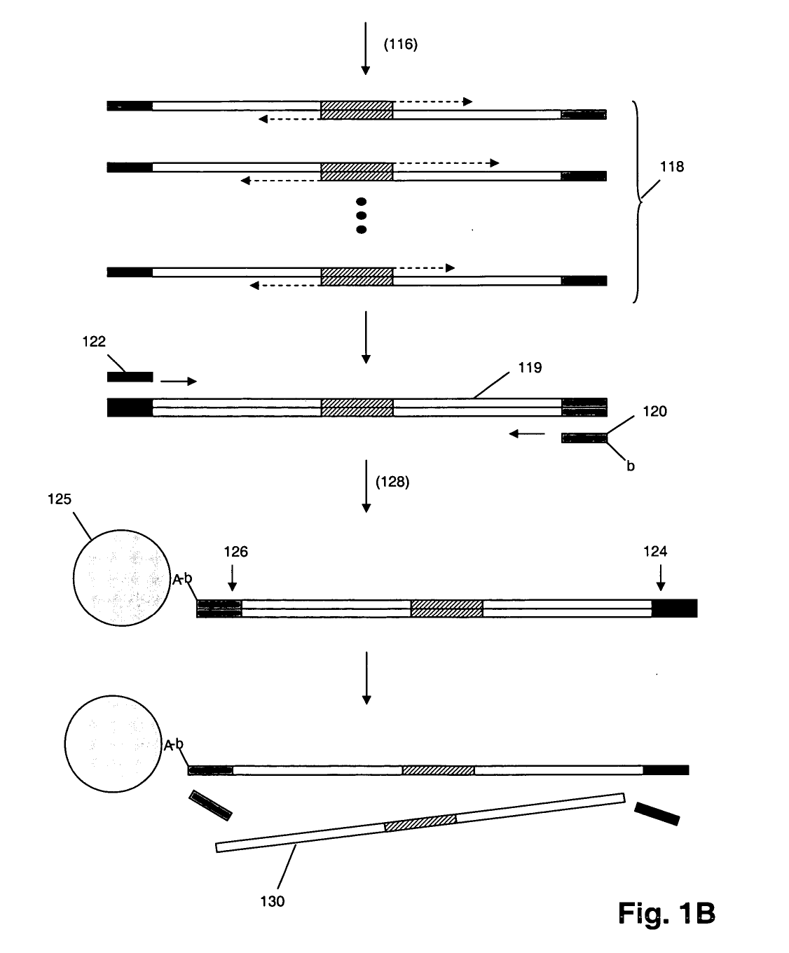

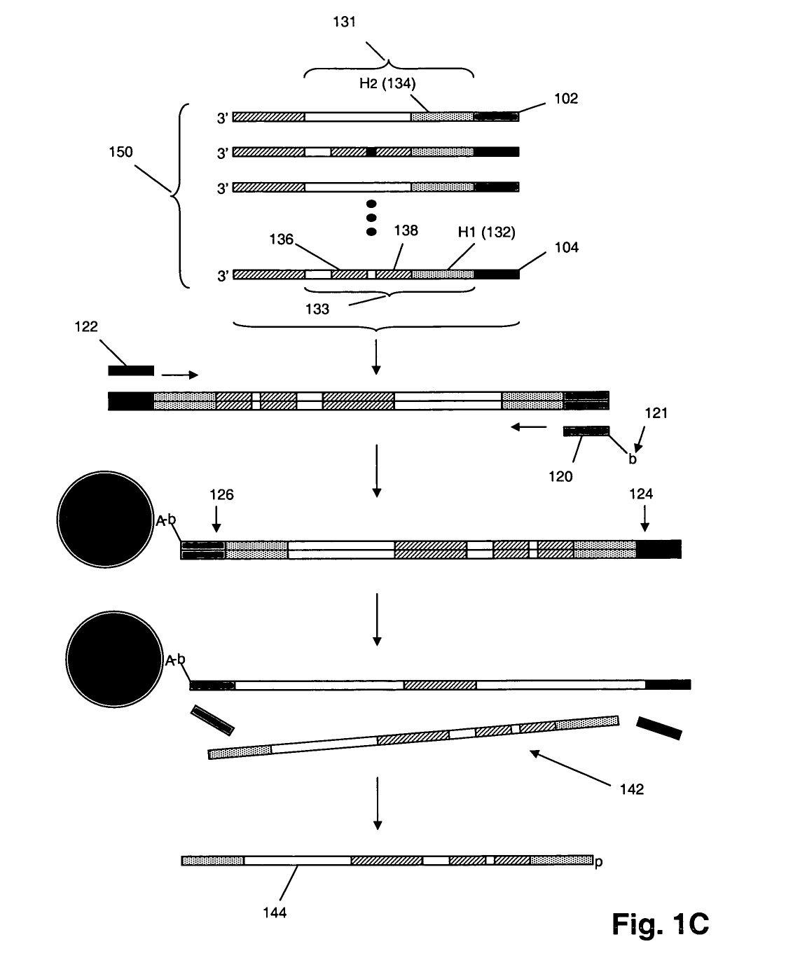

FIGS. 1A-1C illustrate an exemplary embodiment of the invention that uses oligonucleotides synthesized on microarray (100) to produce first and second oligonucleotide mixtures, having common primer binding sites (102) and (104), respectively. In one aspect, such mixtures each have a barcode sequence (106), variable region (105) or (107), and common primer binding sites, “P₁” or (102) for the first oligonucleotide mixture and “P₂” or (104) for the second oligonucleotide mixture. In one aspect of the invention, all oligonucleotides of the first oligonucleotide mixture have the same primer binding site (102); and likewise, all oligonucleotides of the second oligonucletide mixture have the same primer binding site (104). As used herein, the term “primer binding site’ refers either the segment of an oligonucleotide that a primer binds to in an amplification reaction, or its complement, as appropriate. Variable regions (105) and (107) can vary widely in length and composition depending on the intended use of polynucleotides (110) or (130). In one aspect, where variable regions (105) are employed as probes to specifically hybridize to a target polynucleotide, lengths of variable regions (105) and (107) are in the range of from 0 to 100 nucleotides. In another aspect, at least one of either (105) or (107) has a non-zero length suitable for a hybridization probe. In a preferred embodiment, such non-zero length is in the range of from 8 to 60 nucleotide; in another preferred embodiment, such range is from 15 to 40 nucleotides. In another aspect, where polynucleotides (130) are used as circularizing probes, such as MIPs or padlock probes, the lengths of each of variable region (105) and (107) are at least 12 nucleotides and the sum of their lengths is within the range of from 30 to 60 nucleotides.

The first and second oligonucleotides may be synthesized separately or together on the same one or more solid phase supports. The synthesis of high-density microarrays is disclosed in the following exemplary references that are incorporated by reference: Fodor et al, U.S. Pat. Nos. 5,424,186; 5,744,305; 5,445,934; 6,355,432; 6,440,667 (Affymetrix, Santax Clara, Calif.). In particular, the following references (which are incorporated by reference) disclose synthesis and cleavage of mixtures of oligonucleotides from microarrays: Weiler et al, Anal. Biochem., 243: 218-227 (1996); and Lipschutz et al, U.S. Pat. No. 6,440,677. First and second oligonucleotides may be synthesized from either the 3′→5′ direction (with the oligonucleotide attached to the support by its 3′ hydroxyl), or the 5′→3′ direction (with the oligonucleotide attached to the support by its 5′ hydroxyl); however, 3′→5′ synthesis is preferred. Preferably, a solid phase synthesis approach is selected that includes a capping step in each synthesis cycle, so that failure sequences are truncated. This is particularly advantageous when the assembled first and second oligonucleotides are amplified in a polymerase chain reaction, as only successfully completed sequences would have primer binding sites at both ends and thereby be amplified. An important feature of the invention is that cleavage from solid phase supports leave extendable 3′ ends on the oligonucleotides of the first and second oligonucleotide mixtures. Usually, an “extendable end” is a free 3′ hydroxyl group that can be extended by a DNA polymerase in a conventional template-driven extension reaction. As usual for synthesizing an array of polynucleotides, the sequence of each first or second oligonucleotides at each site on a microarray is predetermined; however, in one aspect, such predetermined sequences may include regions of random sequence. That is, regions where one or more consecutive nucleotides are selected at random from the natural nucleotides, or a subset thereof.

Returning to FIG. 1A, after cleavage (110) from solid support (100) (or additional supports, if more than one support is used), first and second oligonucleotide mixtures (108) are subjected (112) to conditions that permit perfectly matched duplexes (114) to form substantially only between complementary barcode sequences. As described more fully below, there is abundant guidance in the literature for establishing such conditions. Construction of sets of barcode sequences, or oligonucleotide tags, is well-known in the art, as are selection of hybridization reaction conditions. Barcode sequences, or equivalently, oligonucleotide tag sequences, may be selected so that substantially all members of a set have the same melting temperature, or duplex stability. Thus, for a selected set of barcode sequences, hybridization reaction conditions may be readily selected so that substantially all barcode sequences and their complements form perfectly matched duplexes. Although not shown in FIG. 1A, it would be clear to one of ordinary skill that there could be regions of complementarity between first and second oligonucleotes in addition to the barcode sequences and their complements. After barcode sequences anneal to their complements, the 3′ ends of the first and second oligonucleotides are extended (116) in a conventional polymerase reaction so that the single stranded portions of the duplexes are filled (118) in to form double stranded DNAs (119). To double stranded DNAs (119) are added primers (122) and (120), one of which is labeled with biotin (121), or like capture moiety, after which double stranded DNA is amplified. The degree of amplification, e.g. the number of cycles if PCR is employed, depends on several factors including, but not limited to, the amount of product required, the complexity of the polynucleotide mixture, the length of the oligonucleotides from which first and second amplicons are made, and the like. For PCR amplifications, usually a conventional reaction of 25-30 cycles is performed in a reaction volume of 50-100 μL. Each mixture of first and second oligonucleotides contains pluralities of different oligonucleotides. In one aspect, the size of such pluralities are determined by several factors, including the multiplexing capacity of the solid phase synthesis, and the size of the set of barcode sequences, and the like. Accordingly, each such plurality may vary widely in different embodiment. For example, embodiments may have pluralities in the range of from 2 to 100,000; or in the range of from 2 to 50,000; or in the range of from 2 to 30,000; or in the range of from 2 to 20,000; or in the range of from 2 to 10,000; or in the range of from 100 to 5,000; or in the range of from 1,000 to 10,000; or in the range of from 1,000 to 50,000; or in the range of from 5,000 to 500,000. The lengths of the oligonucleotide used to make the first and second oligonucleotides may also vary widely. In one aspect, such lengths may be determined by the ability to produce sufficient starting material in the selected synthetic approach to permit subsequent amplification to the desired quantity for hybridization and extension. Or, such lengths may be further determined by to chemistry used to synthesize the oligonucleotides. Lengths of the oligonucleotides used to make the first and second amplicons may be selected in the range of from 18 to 150; or in the range of from 24 to 100; or in the range of from 24 to 75.

After amplification, the resulting amplicon is captured (128) on solid phase support (125), e.g. which may be avidinated magnetic beads. In order to obtain a desired polynucleotide (130), the sequences of primer binding sites (124) and (126) are preferably separable from the desired polynucleotide (130). Primer binding site (124) may be removed by digestion with a restriction enzyme to that it is removed from both strands of the amplicon. Preferably primer binding site (126) is separated only from (130) so that the complement or (130) remains attached to the solid support and can be separated from (130). In one aspect primer binding sites (126) and/or (124) are selected to contain recognition sites for a nicking enzyme. In one aspect a type IIs nicking enzyme may be used, e.g. N.Alw I and/or N.BstNB I (available from New England Biolabs, Beverly, Mass.). Other nicking enzymes that may be used include, for example, Nb. Bsm I, N. BbvC IA and N. BbvC IB. In another aspect the primers are engineered so that primer (120) contains a restriction enzyme recognition site and the site in the primer is blocked from cleavage, for example, by incorporation of a thiol linkage. After capture and washing, single stranded polynucleotide (130) may be release by treating with such nicking enzymes, after which it may be purified from the reaction mixture and solid phase supports by conventional means, e.g. preparative gel electrophoresis. For polynucleotides being used as circularizing probes, 5′ phosphate groups may be added enzymatically using a conventional kinase reaction.

The above process may be used to synthesize circularizable probes, such as molecular inversion probes (MIPs), described more fully below. Such synthesis is illustrated in FIG. 1C. A mixture (150) of first and second oligonucleotides is synthesized and cleaved from solid phase supports as illustrated in FIG. 1A. Variable regions (131) of first oligonucleotides (102) contain regions (134 or “H2”) that are adjacent to first primer binding sites. The sequences of regions (134) are complementary to regions of target nucleic acids. Variable regions (133) of second oligonucleotides (104) contain regions (132 or “H1”) that are adjacent to second primer binding sites. The sequence of region (132) is complementary to a region of a target nucleic acid. Variable regions (133) further include common primer binding sites (136) and (138). Otherwise, the same steps are employed for producing MIPs (144) as described above.

As mentioned above, polynucleotide mixtures of the invention may be employed as circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP 4-262799; rolling circle probes being disclosed in Aono et al, JP-4-262799; Lizardi, U.S. Pat. Nos. 5,854,033; 6,183,960; 6,344,239; molecular inversion probes being disclosed in Hardenbol et al (cited above) and in Willis et al, U.S. Pat. No. 6,858,412; and linear amplification molecules being disclosed in Faham et al, U.S. patent publication 2003/0104459; all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatatemers, and the like, with an exonuclease, such as exonuclease I. As used herein, “padlock probe” means a linear polynucleotide that has target-specific sequences at each end such that a target polynucleotide having complementary sequences to such ends can be detected in a template-driven ligation reaction (which ligation reaction may include a combination of polymerase extension and ligation) that forms a circular DNA molecule. Thus, MIPs are a special cases of padlock probes. As used herein, a “linear ligation probe” means a linear polynucleotide that has a target-specific sequence at at least one end such that a target polynucleotide having a complementary sequence to such end can be detected in a template-driven ligation reaction with another target-specific probe (which ligation reaction may include a combination of polymerase extension and ligation) to form a linear DNA molecule. Examples of linear ligation probes include, but are not limited to, MLPA probes, selector probes, and the like.

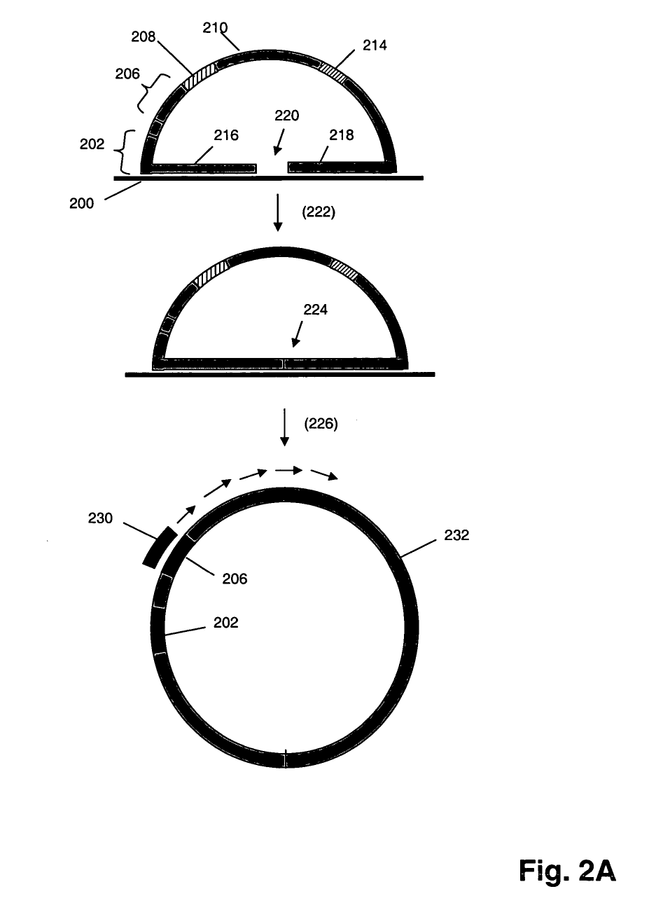

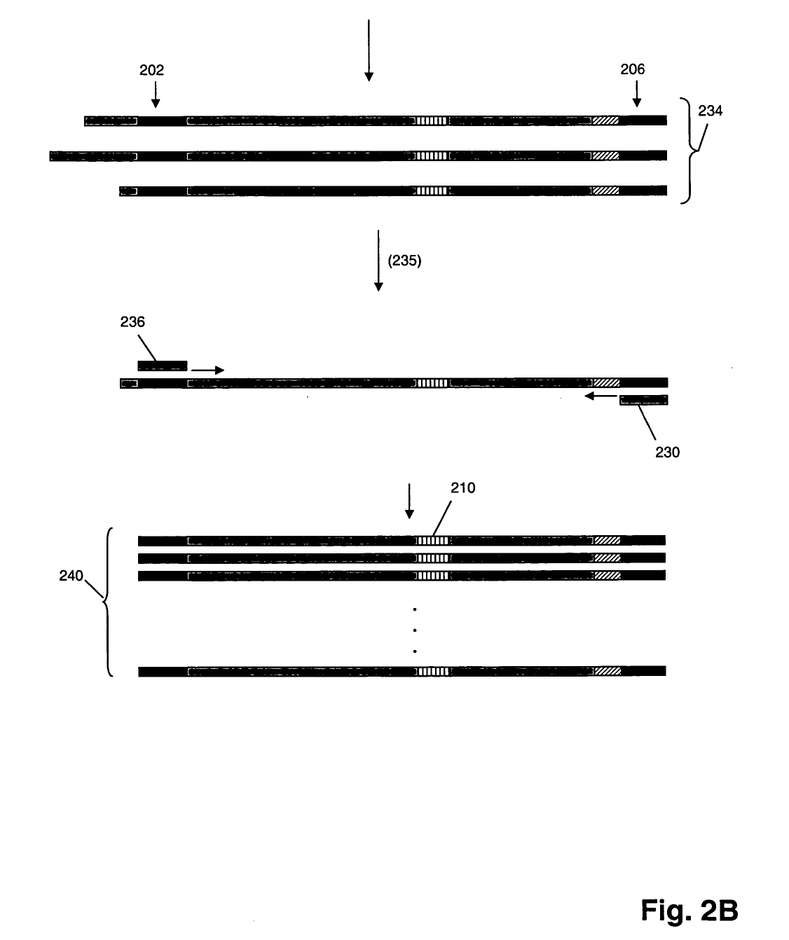

FIG. 2 illustrates a molecular inversion probe and how it can be used to generate an amplicon after interacting with a target polynucleotide in a sample. A linear version of the probe is combined with a sample containing target polynucleotide (200) under conditions that permit target-specific region 1 (216) and target-specific region 2 (218) to form stable duplexes with complementary regions of target polynucleotide (200). The ends of the target-specific regions may abut one another (being separated by a “nick”) or there may be a gap (220) of several (e.g. 1-10 nucleotides) between them. In either case, after hybridization of the target-specific regions, the ends of the two target specific regions are covalently linked by way of a ligation reaction or an extension reaction followed by a ligation reaction. The latter reaction is carried out by extending with a DNA polymerase a free 3′ end of one of the target-specific regions so that the extended end abuts the end of the other target-specific region, which has a 5′ phosphate, or like group, to permit ligation. In one aspect, a molecular inversion probe has a structure as illustrated in FIG. 2. Besides target-specific regions (216 and 218), in sequence such a probe may include first primer binding site (202), optional cleavage site (204), second primer binding site (206), first tag-adjacent sequences (208) (usually restriction endonuclease sites and/or primer binding sites) for tailoring one end of a labeled target sequence containing oligonucleotide tag (or barcode sequence) (210), and second tag-adjacent sequences (214) for tailoring the other end of a labeled target sequence. In operation, after specific hybridization of the target-specific regions and their ligation (222), the reaction mixture is treated with a single stranded exonuclease that preferentially digests all single stranded nucleic acids, except circularized probes. In one embodiment of molecular inversion probes, after such treatment, circularized probes are treated with a cleaving agent that cleaves the probe between primer (202) and primer (206) so that the structure is linearized for PCR amplification. In another embodiment, which is illustrated in FIGS. 2A-2B, circularized probes (232) are not cleaved, instead a single primer (230) common to all probes is annealed and extended (226) to make linear copies (234) of the circularized probes that include at least both primer binding sites (202) and (206). After such copies are made, the second primer (236) is added (235) so that amplicon (240) can be produced by PCR, or like amplification technique. Such amplicons are then detected by conventional techniques, e.g. Willis et al, U.S. Pat. No. 6,858,412, which is incorporated by reference. A multiplexed readout may be obtained from amplicon (240) by labeling and excising oligonucleotide tag (210) and specifically hybridizing the labeled tags to a microarray of tag complements, e.g. a GenFlex array (Affymetrix, Santa Clara, Calif.); a bead array (Illumina, San Diego, Calif.); or a fluid array, e.g. Chandler et al, U.S. Pat. No. 5,981,180 (Lumenix, Austin, Tex.).

### Oligonucleotide Tags and Minimally Cross-Hybridizing Sets

In one aspect, the invention provides a method of oligonucleotide tags, or barcode sequences, to assemble polynucleotide probes. Such tag or barcode sequences may comprise minimally cross-hybridizing sets of oligonucleotide tags, such as disclosed in Brenner et al, U.S. Pat. No. 5,846,719; Mao et al (cited above); Fan et al, International patent publication WO 2000/058516; Morris et al, U.S. Pat. No. 6,458,530; Morris et al, U.S. patent publication 2003/0104436; Church et al, European patent publication 0 303 459; Huang et al, U.S. Pat. No. 6,709,816; which references are incorporated herein by reference. The sequences of oligonucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleoitde tags. In particular, tag complements may comprise peptide nucleic acids (PNAs). Oligonucleotide tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication 2003/0104436; and Huang et al (cited above). The term “oligonucleotide tag” is used interchangeably with the term “barcode,” or “barcode sequence.”

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), e119 (2003).

Preferably, oligonucleotide tags and tag complements are selected to have similar duplex or triplex stabilities to one another so that perfectly matched hybrids have similar or substantially identical melting temperatures. This permits mis-matched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like. A minimally cross-hybridizing set of oligonucleotides may be screened by additional criteria, such as GC-content, distribution of mismatches, theoretical melting temperature, and the like, to form a subset which is also a minimally cross-hybridizing set.

### Hybridization of Labeled Target Sequence to Solid Phase Supports

Methods for hybridizing labeled target sequences (such as amplified and labeled barcode sequences from MIPs) to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled target sequence to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂,PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3M TMACL (Tetramethylammonium. Chloride), 50 mM MES ((2-[N-Morpholino]ethanesulfonic acid) Sodium Salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip Software (Affymetrix) may be used to convert the image files into digitized files for further data analysis.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

What is claimed is:

1. A method of synthesizing a mixture of polynucleotides, the method comprising the steps of:
(a) synthesizing a plurality of first oligonucleotides on a first microarray, each first oligonucleotide having a predetermined sequence comprising in the 3′ to 5′ direction a first barcode sequence, a first variable region, and a first primer binding site;
(b) synthesizing a plurality of second oligonucleotides on a second microarray, each second oligonucleotide having a predetermined sequence comprising in the 3′ to 5′ direction a second barcode sequence, and a second primer binding site, the second barcode sequences being selected so that for every first barcode sequence there is at least one second barcode sequence complementary thereto;
(c) cleaving the first oligonucleotides and the second oligonucleotide from the first and second microarrays so that such cleaved first oligonucleotides and second oligonucleotides have extendable 3′ ends;
(d) mixing the cleaved first oligonucleotides and second oligonucleotides under conditions that permit the formation of stable duplexes substantially only between first barcode sequences and complementary second barcode sequences; and
(e) extending 3′ ends of the stable duplexes with a DNA polymerase to form a mixture of polynucleotides, each polynucleotide of the mixture having first and second primer binding sites.

2. The method of claim 1 wherein said first microarray and said second microarray are the same.

3. The method of claim 1 wherein said barcode sequences are members of a minimally cross-hybridizing set of oligonucleotides.

4. The method of claim 3 further including the steps of amplifying said extended polynucleotides in said mixture to form an amplicon, removing said first and second primer binding sites, and isolating said polynucleotide.

5. The method of claim 4 wherein said step of amplifying is carried out with a polymerase chain reaction using at least one primer that is specific for either said first primer binding site or said second primer binding site and that has a capture moiety attached.

6. The method of claim 5 wherein said step of removing includes the steps of capturing said amplicon on a solid phase support by said capture moiety, cleaving said first and second primer binding sites from a strand of said amplicon, melting said polynucleotide from said amplicon, and separating said polynucleotide from said first and second primer binding sites and said solid phase support.

7. The method of claim 6 wherein said second oligonucleotide has a second variable region and said polynucleotide is a molecular inversion probe or a padlock probe and wherein said step of separating further includes phosphorylating 5′ ends of said polynucleotides.

8. The method of claim 6 wherein said polynucleotide is a linear ligation probe and wherein said step of separating further includes phosphorylating 5′ ends of said polynucleotides.

9. The method of claim 6 wherein said cleaving comprises digestion with a nicking restriction enzyme.

10. The method of claim 9 wherein said nicking restriction enzyme is selected from the group consisting of N. BstNB I and N. Alw I.

11. The method of claim 5 wherein said capture moiety comprises biotin.

12. The method of claim 1 wherein said mixture of polynucleotides comprises between 1,000 and 10,000 different polynucleotides.

13. The method of claim 1 wherein said first variable region comprises a third primer binding site and a fourth primer binding site and a first target complementary sequence.

14. The method of claim 13 wherein said second oligonucleotide comprises a second target complementary sequence and wherein said first and second target complementary sequences are complementary to adjacent target regions.
